(19) United States
(12) Patent Application Publication
(10) Pub. No.: US 2006/0147406 A1
(43) Pub. Date: Jul. 6, 2006

(54) Viscous products for use on the body

(76) Inventors: Patrick T. Yerby, Woodstock, IL (US); Mort Westman, Oak Brook, IL (US)

Correspondence Address: WOOD, PHILLIPS, KATZ, CLARK & MORTIMER, 500 W. MADISON STREET, SUITE 3800, CHICAGO, IL, 60661, US

(21) Appl. No.: 11/336,025

(22) Filed: Jan. 20, 2006

Related U.S. Application Data
(63) Continuation-in-part of application No. 11/188,338, filed on Jul. 25, 2005.
(60) Provisional application No. 60/600,965, filed on Aug. 12, 2004.

(51) Int. Cl. A61K 8/46

(52) U.S. Cl. 424/70.24; 424/401

(57) ABSTRACT

Viscous products for use on the body are formulated from a first low viscosity component formulation and a second low viscosity component formulation. The viscous products can be post-foaming and heated upon reacting the first low viscosity component formulation with the second low viscosity component formulation. The viscous products are formulated for shaving, body washing, tanning, skin or hair conditioning or rinsing. A dispenser contains the viscous products.

### CROSS REFERENCE TO RELATED APLICATION

This application is a continuation-in-part of application Ser. No. 11/188,338, filed Jul. 25, 2005 which claims the benefit of U.S. provisional patent application Ser. No. 60/600,965, filed Aug. 12, 2004.

### TECHNICAL FIELD

The invention relates to viscous products prepared from two low viscosity component formulations. The viscous products can be self-heated and/or post-foaming. The viscous products are formulated for various uses such as shaving, body washing, tanning, skin or hair conditioning or rinsing. A dispenser contains the two low viscosity component formulations.

### BACKGROUND OF THE INVENTION AND TECHNICAL PROBLEMS POSED BY THE PRIOR ART

Compositions are known in the art for shaving, skin conditioning or hand and body cleansing and conditioning, tanning, moisturizing, hair conditioning or facial cleansing. Dispensers for these compositions may be used to contain the compositions prior to use. While the use of dual dispensing aerosol systems to simultaneously dispense two product components is known, it has not been possible to dispense the product in a viscous form.

For example, U.S. Pat. No. 3,250,680 discloses a heat-generating cosmetic composition. The cosmetic composition is in the form of a liquid, cream, gel or paste which is adapted to evolve a sensible quantity of heat when it contacts moisture. The composition is provided by dispersing in a liquid vehicle a quantity of finely divided solid absorbent materials capable of absorbing water and producing an exothermic reaction. The composition is prepared by mixing all of the ingredients together at room temperature.

U.S. Pat. No. 3,341,418 discloses a self-heating shaving composition which comprises a two-part composition adapted to be mixed immediately prior to or during application. One part of the composition contains an oxidant and the other contains a reductant reactive therewith upon mixing to evolve heat. The compositions are contained in a pressurized package having one compartment for each components of the two-part composition and a gaseous propellant for expelling the components of the composition from the compartments.

Self-heating compositions such as aqueous shaving creams are disclosed in U.S. Pat. No. 3,585,982. At least one of the components of a heat-generating combination is encapsulated in a rupturable capsule and dispersed in the composition. The capsule may have a melting point at or below the temperatures which will be generated in the composition.

Self-heating systems for use in producing heated compositions such as shaving creams, comprising an oxidant and a reductant are disclosed in U.S. Pat. No. 3,878,118. The reductant is a substituted glyoxylic acid or salt thereof.

U.S. Pat. No. 4,439,416 discloses a cosmetic preparation that is heated at the time of use by an exothermic reaction. The self-heating cosmetic preparation comprises a hydride and a reducible material that reacts with the hydride which are packaged in a separated pressure-tight dispenser. A propellant is used to eject the contents of the separate containers in the dispenser.

International Patent Publication WO 01/64176 discloses cleansing gels that release heat when mixed with water. The gels contain at least 40 weight percent of hydroxyl compounds that can be mixed with water, at least 5 weight percent of surfactants, at least 5 weight percent of dispersed water-soluble salts having negative solution enthalpy in water, and at least 0.1 weight percent of a dissolved water-soluble thickener or at least 1 weight percent of a dispersed particle-shaped inorganic thickener.

U.S. Pat. No. 5,538,720 discloses a hair treatment composition. The hair treatment composition comprises at least two components that are kept separate until application and that upon mixing, increase in viscosity while generating heat.

U.S. Pat. No. 3,240,396 discloses an aerosol dispenser with an outer dispenser that comprises a single valve actuatable by pressure applied externally of a casing assembly. A hydrophilic thermogenic agent is provided in the component formulation chamber for reaction with water to produce a thermogenic reaction.

### SUMMARY OF THE INVENTION

The invention relates to viscous products for application to the body prepared from two low viscosity component formulations.

Preferably, the viscous products are heated and post-foaming.

Preferably, the viscous products are formulated for shaving, body washing, tanning, skin or hair conditioning or rinsing.

In one preferred embodiment of the invention, the viscous products generate heat, while self-thickening, without the presence of hydrogen peroxide.

The viscous products are applied from a dispenser, preferably, the dispenser disclosed in U.S. Patent Publication No. 2004/0004088, which is incorporated by reference herein.

### DESCRIPTION OF THE PREFERRED EMBODIMENTS

While this invention is susceptible of embodiment in many different forms, this specification and the accompanying examples disclose only some specific forms as examples of the invention. The invention is not intended to be limited to the embodiments so described, however. The scope of the invention is pointed out in the appended claims.

Viscous products for use on the body are produced by the reaction of two low viscosity component formulations that, preferably, produce a self-thickening, exothermic, post-foaming product.

The low viscosity component formulations each comprise a separate phase with different compositions. The compositions and their percent weight are selected based on the end uses of the viscous products such as for shaving, body washing, tanning, skin or hair conditioning or rinsing. The first component formulation usually comprises a surfactant and a reducing agent. The second component formulation usually comprises a diluent and a thickener and is kept separate from the first component formulation. When mixed together with the first component formulation, a thick, warm highly viscous material is formed. A low boiling material may be incorporated to create post-foaming.

For a viscous product that is used for shaving, the first low viscosity component formulation can contain a diluent or solvent, a wetting agent or foam generator, a material that thickens and adds lubricity to the foam, a solvent or humectant, a material that generates heat, a material that provides the low viscosity of the first component formulation and thickens the foam that is created when reacted with the second low viscosity component formulation, a material that increases the pH of the polymer in the second low viscosity component formulation, and a fragrance. Additionally, the first low viscosity component formulation can have a preservative. The second low viscosity component formulation should at least have a diluent or solvent, and a thickener and preferably a material that generates heat when combined with the first low viscosity component formulation.

Alternatively, for formulations that do not contain hydrogen peroxide, the second low viscosity component formulation contains a diluent or solvent, an acidifier, a stabilizer and a thickener.

For a viscous product that is used for body washing, the first low viscosity component formulation can contain a diluent or solvent, a wetting agent or foam generator, a material that thickens and adds lubricity to the foam, a solvent or humectant, a material that generates heat, a material that provides the low viscosity of the first component formulation and thickens the foam, a solvent to ensure homogeneity, a material for imparting a lubricious/conditioned after-feel on the skin, a material to increase the pH of the polymer in the second low viscosity component formulation, a preservative and a fragrance. The second low viscosity component formulation should at least have a diluent or solvent, a material that generates heat, a thickener, a solubilizer and an opacifier.

For a viscous product that is used for tanning, the first low viscosity component formulation can contain a diluent or solvent, a wetting agent or foam generator, a material that thickens and adds lubricity to the foam, a solvent or humectant, a material that generates heat, a material that adds slip during application, such as an emollient, an active ingredient that causes tanning, a solvent to ensure homogeneity, a material that decreases the pH while thickens the polymer in the second low viscosity component formulation, a preservative, an indicator of where the viscous product has been applied and to enhance the color of the tan and a fragrance. The second low viscosity component formulation should at least have a diluent or solvent, a thickener, a solubilizer, an opacifier, a preservative and a material that provides rapid tanning.

For a viscous product that is used for skin conditioning, the first low viscosity component formulation can contain a diluent or solvent, a wetting agent or foam generator, a material that thickens and adds lubricity to the foam, an emulsifier, a solvent or humectant, a material that generates heat, a material that provides the viscosity of the first component formulation and thickens the foam, a material that increases the pH while thickening the polymer in the second low viscosity component formulation, a preservative and a fragrance. The second low viscosity component formulation should at least have a diluent or solvent, a thickener and a material that generates heat when combined with the first low viscosity component formulation.

For a viscous product for hair conditioning and rinsing, the first low viscosity component formulation can contain a diluent or solvent, a material that provides general conditioning while suppressing flyaway hair, a solubilizer and wetting agent, a conditioning agent, a material that thickens and adds lubricity to the foam, a solvent or humectant, a material that generates heat, a material that increases the pH while thickening the polymer in the second low viscosity component formulation, a preservative and a fragrance. The second low viscosity component formulation should at least have a diluent or solvent, a thickener and a material that generates heat when combined with the first low viscosity component formulation.

For an exothermic post-foaming shaving gel, the preferred components of the first low viscosity component formulation are as follows: The diluent and/or solvent is preferably distilled or deionized water. The wetting agent and/or foam generator can be an anionic surfactant, soap or nonionic surfactant such as alkyl polyglucoside, preferably sodium lauryl sulfate with a concentration from 0.5 to 90.0%, and preferably from 9.0 to 20.0% (all % are in weight/volume herein). The material for thickening and lubricity can be cetyl alcohol, cetearyl alcohol, lauryl alcohol and stearyl alcohol, among others, preferably lauryl alcohol with a concentration from 0.1 to 7.0%, preferably from 0.2 to 2.0%. The solvent and/or humectant can be propylene glycol, glycerin, sorbitol, 1,4-butanedial or butylene glycol, preferably propylene glycol with a concentration from 0.1 to 15.0%, preferably from 3.0 to 7.0%. The material for generating heat and oxidation by hydrogen peroxide is preferably monoethanolamine sulfite, with a concentration from 0.5 to 30.0%, preferably from 10.0 to 20.0%. The material for providing low viscosity can be an amine oxide, amphoteric or salt, preferably oleamine oxide with a concentration from 0.1 to 15.0%, preferably from 1.0 to 8.0%. The material for increasing the pH and for thickening the polymer in the second low viscosity component formulation can be a base such as an alkyl amine, AMP (2-amino-2-methyl-1-propanol) or an inorganic base, preferably triethanolamine with a concentration from 1.0 to 15.0%, preferably from 3.0 to 8.0%. The fragrance can be present in a concentration from 0.005 to 3.0%, preferably from 0.01 to 1.5%. For the second low viscosity component formulation, the preferred components are as follows: The diluent and/or solvent is preferably distilled or deionized water; the material for generating heat while oxidizing the monoethanolamine sulfite is preferably 6% hydrogen peroxide with a concentration from 10.0 to 80.0%, preferably from 25.0 to 55.0%; and the second low viscosity component formulation preferably contains an acrylate/steareth-20 itaconate copolymer with a concentration from 2.0 to 30.0%, preferably from 5.0 to 25.0%.

The elimination of hydrogen peroxide in exothermic post foaming shaving gels is important in certain countries where regulations do not permit its use in such formulations or where its direct contact with skin is of concern or where handling concerns make its elimination desirable.

For an non-hydrogen peroxide containing exothermic post-foaming shaving gel, the preferred components of the first low viscosity component formulation or base are as follows: the humectant and/or solvent is propylene glycol, glycerin, sorbitol or butylene glycol, preferably propylene glycol, with a concentration from 25.0 to 75.0%, and preferably from 40.0 to 60.0%; the material for thickening and lubricity can be cetyl alcohol, lauryl alcohol, cetearyl alcohol or stearyl alcohol, preferably cetyl alcohol, with a concentration of from 0.05 to 7.0%, preferably from 0.2 to 2.0%; the material that generates heat upon dissolution in water can be a polyalkylene glycol such as polyethylene glycol or a polyalkylene glycol monobutyl ether, a PPG/PEG or PPG/Buteth copolymer, aluminum silicate or combinations thereof, with a concentration of from 10.0 to 7.0%, preferably from 40.0 to 60.0%; and the material that increases and buffers pH can be a base such as triethanolamine, 2-amino-2-methyl-1,2-propanediol, tromethamine, 2-amino-2-methyl-1-propanol, or combinations thereof, with a concentration of from 0.2 to 15.0%, preferably from 3.0 to 8.0%; and the fragrance can be present in a concentration from 0.005 to 3.0%, preferably from 0.1 to 1.5%. The second low viscosity component formulation or developer preferably contains the following: the diluent and/or solvent is preferably water, the acidifier is preferably phosphoric acid with a concentration from 0.05 to 7.0%, preferably from 0.2 to 3.0%; any suitable solubilizer such as nonoxynol-4, nonoxynol-9, decyl glucoside, or combinations thereof with a concentration from 1.0 to 18.0%, preferably from 3.0 to 12.0%; and a thickener such as an acrylate/steareth-20 itaconate copolymer with a concentration from 2.0 to 30.0%, preferably from 5.0 to 25.0%.

For an exothermic post-foaming body wash gel, the preferred components for the first low viscosity component formulation are as follows: The diluent and/or solvent is preferably distilled or deionized water. The wetting agent and/or foam generator can be an anionic surfactant, soap or nonionic surfactant such as an alkyl polyglucoside, preferably sodium lauryl sulfate with a concentration from 0.5 to 90.0%, preferably from 9.0 to 20.0% (all % are in weight/volume herein). The material that thickens and provides lubricity to the foam can be lauryl alcohol, cetyl alcohol, cetearyl alcohol or stearyl alcohol, among others, preferably lauryl alcohol with a concentration from 0.1 to 7.0%, preferably from 0.2 to 2.0%. The solvent and/or humectant can be glycerin, butylene glycol, sorbitol, 1,4-butanedial or propylene glycol, preferably glycerin with a concentration from 0.1 to 15.0%, preferably from 3.0 to 7.0%. The material that generates heat upon oxidation by hydrogen peroxide can be monoethanolamine sulfite with a concentration from 0.5 to 30.0%, preferably from 10.0 to 20.0%. The material that provides low viscosity to the first component formulation can be an amine oxide, amphoteric surfactant, or salt, preferably cocamidopropyl betaine with a concentration from 0.1 to 15.0%, preferably from 1.0 to 8.0%. The solvent for ensuring homogeneity can be ethanol, isopropanol or propylene glycol, preferably ethanol with a concentration from 0.1 to 7.5%, preferably from 0.5 to 5.0%. The material that imparts lubricity and/or a conditioned after-feel to the skin can be a cationic polymer, preferably Polyquaternium-10 with a concentration from 0.05 to 2.5%, preferably from 0.05 to 1.0%. The material that increases the pH to thicken the polymer in the second low viscosity component formulation can be a base such as an alkyl amine, AMP (2-amino-2-methyl-1-propanol) or an inorganic base, preferably triethanolamine with a concentration from 1.0 to 15.0%, preferably from 3.0 to 8.0%. A microbiological preservative can be added such as KATHON CG™ from Rohm & Hass or GERMALL PLUS™ from ISP, preferably DMDM hydantoin with a concentration from 0.05 to 2.0%, preferably from 0.1 to 1.0%. For the fragrance, numerous materials can be used, and the concentration can be from 0.005 to 3.0%, preferably from 0.01 to 1.5%. For the second low viscosity component formulation, the preferred components are as follows: The diluent and/or solvent is preferably distilled or deionized water. The material that generates heat upon oxidizing the monoethanolamine sulfite is preferably 6% hydrogen peroxide with a concentration from 10.0 to 80.0%, preferably from 25.0 to 55.0%. An agent that causes thickening in combination with the first low viscosity component formulation, preferably an acrylate/steareth-20 itaconate copolymer with a concentration from 2.0 to 30.0%, preferably from 5.0 to 25.0%. Solubilizers such as nonoxynol-4, nonoxynol-9, and decyl glucoside, all with a concentration from 0.05 to 5.0%, preferably 0.01 to 3.0% are presently used. Ingredients such as cetyl alcohol or stearyl alcohol can be used to thicken, opacify, and impart a lubricious feel to the lather and to skin.

For a post-foaming quick self-tanning gel, the preferred components for the first low viscosity component formulation are as follows: The diluent and/or solvent is preferably distilled or deionized water. The wetting agent and/or solubilizer can be a nonionic surfactant, preferably octoxynol-9. The material that thickens and provides lubricity to the foam can be lauryl alcohol, cetyl alcohol, cetearyl alcohol or stearyl alcohol, preferably lauryl alcohol with a concentration from 0.1 to 7.0%, preferably from 0.2 to 2.0%. The solvent and/or humectant can be propylene glycol, butylene glycol, 1,4-butanediol, or sorbitol, preferably propylene glycol with a concentration from 0.1 to 15.0%, preferably from 3.0 to 7.0%. The material that provides slip during application and is used as an emollient is preferably dimethicone copolyol. The solvent for ensuring homogeneity can be ethanol, isopropanol or propylene glycol, preferably ethanol with a concentration from 0.1 to 7.5%, preferably from 0.5 to 5.0%. The active ingredient for tanning is preferably dihydroxyacetone with a concentration from 2.0 to 22.0%, preferably from 6.0 to 18.0%. The material that decreases the pH and/or thickens the polymer in the second component formulation is preferably citric acid, but there are numerous alternate acids such as phosphoric acid, hydrochloric acid and lactic acid that can be used. The concentration of the citric acid can be from 0.01 to 5.0%, preferably from 0.05 to 1.5%. Suitable preservatives for maintaining product bio-integrity are methylparaben and propylparaben, but alternates that do not contain amines can be used. The preservatives can have a concentration from 0.20 to 1.0%, preferably from 0.05 to 1.0%. An indicator of where the product has been applied and to enhance the color of the skin that is used is preferably caramel, but similar alternates are carotene, D&C Blue No. 4 and D&C Red No. 21, among others. The concentration of the caramel can be from 0.01 to 4.0%, preferably from 0.1 to 2.0%. For the fragrance, numerous alternates can be used, and the concentration can be from 0.005 to 4.0%, preferably from 0.05 to 1.5%. For the second low viscosity component formulation, the preferred components are as follows: The diluent and/or solvent is preferably distilled or deionized water. An agent that causes thickening in combination with the first low viscosity component formulation, preferably an acrylate/steareth-20 itaconate copolymer is used in a concentration from 2.0 to 30.0%, preferably from 5.0 to 25.0%. The material for rapid coloration and tanning is preferably glycine, but histidine and other amino acids can be used. The glycine can have a concentration from 0.1 to 15.0%, preferably from 1.0 to 5.0%. While numerous nonionic surfactants may be employed, nonoxynol-9, and decyl glucoside are the preferred solubilizer, opacifier and/or thickener, and all can have a concentration from 0.05 to 5.0%, preferably 0.01 to 3.0%. Fatty alcohols such as cetearyl alcohol, stearyl alcohol and cetyl alcohol may also be used to opacify and thicken the second low viscosity component formulation. Two preferred microbiological preservatives for maintaining product bio-integrity are methylparaben and propylparaben. Alternative materials that do not contain amines may be used. The concentration of preservative can be from 0.02 to 1.0%, preferably from 0.05 to 1.0%

For an exothermic skin gel, the preferred components for the first low viscosity component formulation are as follows: The diluent and/or solvent is preferably distilled or deionized water. The wetting agent and/or solvent can be an anionic surfactant, soap or nonionic surfactant such as an alkyl polyglucoside, preferably ceteareth-25 with a concentration from 5.0 to 25.0%, preferably from 9.0 to 18.0%. The material for thickening and adding lubricity to the foam can be lauryl alcohol, cetyl alcohol, cetearyl alcohol or stearyl alcohol, preferably lauryl alcohol with a concentration from 0.1 to 7.0%, preferably from 0.2 to 2.0%. The emulsifier and/or solubilizer can be a nonionic surfactant, preferably PEG-25 (polyethylene glycol-25) hydrogenated castor oil with a concentration from 0.2 to 10.0%, preferably from 0.5 to 5.0%. The solvent and/or humectant can be glycerin, propylene glycol, sorbitol, 1-4 butanediol, or butylene glycol, preferably propylene glycol with a concentration from 0.1 to 15.0%, preferably from 3.0 to 7.0%. The material for generating heat when oxidized by the hydrogen peroxide of the second component formulation, is preferably monoethanolamine sulfite with a concentration from 0.5 to 30.0%, preferably from 10.0 to 20.0%. The material for providing low viscosity to the first component formulation and for thickening the foam can be an amine oxide, amphoteric or salt, preferably cocamidopropyl betaine with a concentration from 0.1 to 15.0%, preferably from 1.0 to 8.0%. The material for increasing the pH and hence thicken the polymer in the second low viscosity component formulation can be a base such as an alkyl amine, AMP (2-amino-2-methyl-1-propanol) or an inorganic base, preferably triethanolamine with a concentration from 1.0 to 15.0%, preferably from 3.0 to 8.0%. The microbiological preservative is preferably DMDM hydantoin and can have a concentration from 0.05 to 2.0%, preferably from 0.1 to 1.0%. Numerous fragrances can be used with a concentration from 0.005 to 3.0%, preferably from 0.01 to 1.5%. For the second low viscosity component formulation, the preferred components are as follows: The diluent and/or solvent is preferably distilled or deionized water. The material for generating heat while oxidizing the monoethanolamine sulfite is preferably 6% hydrogen peroxide with a concentration from 10.0 to 80.0%, preferably from 25.0 to 55.0%. The second low viscosity component formulation contains an agent to cause thickening in combination with the first component formulation and preferably contains an acrylate/steareth-20 itaconate copolymer with a concentration from 2.0 to 30.0%, preferably from 5.0 to 25.0%.

For an exothermic post-foaming hair conditioning gel, the preferred components for the first low viscosity component formulation are as follows. The diluent and/or solvent is preferably distilled or deionized water. The material for general conditioning and suppressing flyaway can be a quaternary ammonium surfactant, preferably cetrimonium chloride with a concentration from 0.1 to 8.0%, preferably from 0.5 to 5.0%. The material for improving wet and dry comb and conditioning hair can be a cationic polymer, preferably polyquaternium-10 with a concentration from 0.05 to 2.5%, preferably from 0.05 to 1.0%. The solubilizer or wetting agent can be a nonionic surfactant, preferably PEG-7 (polyethylene glycol-27) glyceryl cocoate with a concentration from 0.10 to 7.5%, preferably from 0.2 to 4.0%. The conditioning agent can be a source of protein, preferably quaternized wheat protein with a concentration from 0.05 to 10.0%, preferably from 0.1 to 5.0%. The material for thickening and adding lubricity to the foam can be lauryl alcohol, cetyl alcohol, cetearyl alcohol or stearyl alcohol, preferably lauryl alcohol with a concentration from 0.1 to 7.0%, preferably from 0.2 to 2.0%. The solvent and/or humectant can be glycerin, propylene glycol, 1,4-butantediol, sorbitol or butylene glycol, preferably propylene glycol with a concentration from 0.1 to 15.0%, preferably from 3.0 to 7.0%. The material for generating heat when it is oxidized by the second low viscosity component formulation is preferably monoethanolamine sulfite with a concentration from 0.5 to 30.0%, preferably from 10.0 to 20.0%. The material for increasing the pH in order to thicken the polymer of the second low viscosity component formulation is a base such as an alkyl amine, AMP (2-amino-2-methyl-1-propanol) or an inorganic base, preferably triethanolamine with a concentration from 1.0 to 15.0%, preferably from 3.0 to 8.0%. The microbiological preservative can be KATHON CG™ from Rohm & Hass or GERMALL PLUS™ from ISP, preferably DMDM hydantoin with a concentration from 0.05 to 2.0%, preferably from 0.1 to 1.0%. Numerous fragrances can be used in a concentration from 0.05 to 2.0%, preferably from 0.01 to 1.0%. For the second low viscosity component formulation, the preferred components are as follows: The diluent and/or solvent is preferably distilled or deionized water. The material for generating heat as the result of oxidizing the monoethanolamine sulfite is preferably 6% hydrogen peroxide with a concentration from 10.0 to 80.0%, preferably from 25.0 to 55.0%. The second low viscosity component formulation contains an agent that thickens when combined with the first low viscosity component formulation, preferably an acrylate/steareth-20 itaconate copolymer with a concentration from 2.0 to 30.0%, preferably from 5.0 to 25.0%.

The first and second low viscosity component formulation are mixed together to form the viscous product by using a dispenser having a co-dispensing valve system, preferably that disclosed in U.S. patent application Ser. No. 10/441,568, filed on May 20, 2004, which is published as U.S. Patent Publication No. US 2004/004088, published on Jan. 8, 2004, which claims benefit of priority from U.S. Provisional Application No. 60/382,283, filed on May 21, 2002, and incorporated by reference herein.

The invention is exemplified through the representative examples as described herein. In the examples, the Base is the first low viscosity component formulation and the Developer or Accelerator is the second low viscosity component formulation. The examples provide the trade names of the materials, the suppliers and the percent weight of each composition. A number of the materials may be provided by numerous suppliers.

### EXAMPLE 1

An exothermic post-foaming shaving gel is prepared from the following Base and Developer. The listed concentration is percent by weight.

Base:

1) distilled or deionized water—50.99%, 2) sodium lauryl sulfate (28% active)—STANDAPOL® WAQ SPECIAL from Cognis Corporation, 14.00%,

3) lauryl alcohol—CO-1214 from Procter & Gamble, 0.75%,

4) propylene glycol—PROPYLENE GLYCOL USP™ from Lyondell Chemical Co., 5.00%,

5) monoethanolamine sulfite—THIOSET M™ from Dow Chemical, 14.26%,

6) oleamine oxide—STANDAMOX 01™ from Cognis Corporation, 4.00%,

7) triethanolamine—from Dow Chemical, 6.30%,

8) fragrance—BELL 6106533™ from Bell Flavors and Fragrances, Inc., 0.06%, and

9) distilled or deionized water—4.44%.

The distilled or deionized water is heated and combined with the sodium lauryl sulfate at a temperature of 50° C. with moderate stirring. Next, lauryl alcohol, polypropylene glycol, monoethanolamine sulfite and oleamine oxide are added. The resulting mixture is cooled to a temperature of 40° C. and 75% of the triethanolamine is added. The resulting mixture is then cooled to 25° C., fragrance is added and the pH is determined. Additional triethanolamine is added as needed to adjust the pH to 9.0, +/−0.2. Water is finally added to bring the weight percent to 100.0%.

Developer:

1) distilled or deionized water—50.00%,

2) 6% hydrogen peroxide (v/v)—hydrogen peroxide and water. Hydrogen peroxide from FMC Corporation Pharmaceutical Div., 40.00%, and

3) acrylate/steareth-20 itaconate copolymer—STRUCTURE® 2001 from National Starch & Chemical Co., 10.00%.

The compositions of the Developer are combined at ambient conditions.

The Base and the Developer are filled in a two-part dispenser having a co-dispensing valve system for mixing and dispensing the Base and the Developer, preferably as described in U.S. Patent Publication No. US 2004/004088. This dispenser allows for simultaneous mixing and dispensing of the base and developer or accelerator.

During filling, 120.0 g of the Base is combined with 10.0 g of isopentane (alternates are low boiling hydrocarbons including pentanes, hexanes, heptanes, among others) while 80.0 g of the Developer are used. This amount is preferred for the ratio dependent orifices of the dispenser.

### EXAMPLE 2

An exothermic post-foaming body wash gel is prepared from the following Base and Developer. The listed concentration is percent by weight

Base:

1) distilled or deionized water—32.69%,

2) sodium lauryl sulfate (28% active)—Standapol® WAQ SPECIAL from Cognis Corporation, 12.00%,

3) sodium laureth sulfate (26% active)—Standapol® ES-2 from Cognis Corporation, 12.00%,

4) lauryl alcohol—CO-1214 from Procter & Gamble, 0.75%,

5) glycerin—glycerin USP from Cognis Corporation, 5.00%,

6) monoethanolamine sulfite—Thioset M™ from Dow Chemical, 14.26%,

7) cocamidopropyl betaine—Monateric™ CAB from Uniqema Americas, 10.00%,

8) ethanol denat. (190 proof)—ethanol SD 40 (190), 2.00%,

9) polyquaternium-10—Ucare™ Polymer JR 400 from Dow Corporation, 0.500%,

10) triethanolamine—from Dow Chemical, 6.30%, used to increase the pH and to thicken the polymer in the Developer,

11) DMDM hydantoin—Glydant® from Lonza, Inc., 0.30%,

12) fragrance—Bell 6106533™ from Bell Flavors and Fragrances, Inc., 0.06%, and

13) distilled or deionized water—4.14%.

The Base is mixed and prepared in a similar manner to Example 1.

Developer:

1) distilled or deionized water—48.20%,

2) 6% hydrogen peroxide (v/v)—hydrogen peroxide and water. Hydrogen peroxide from FMC Corporation Pharmaceutical Div., 40.00%,

3) acrylate/steareth-20 itaconate copolymer—Structure® 2001 from National Starch & Chemical Co., 10.00%,

4) nonoxynol-4—Igepal® CO-430 from Rhodia Inc., 0.50%,

5) nonoxynol-5—Igepal® CO-630 from Rhodia Inc., 0.50%,

6) cetyl alcohol—Lanette® from Cognis Corporation, 0.30%, and

7) decyl glucoside—Plantaren® PS-100 from Cognis Corporation, 0.50%.

The Developer is combined in a similar manner to that described in Example 1. The Base and the Developer are stored and dispensed in a similar manner to that of Example 1.

### EXAMPLE 3

A post-foaming quick self-tanning gel is prepared from the following Base and Accelerator. The listed concentration is percent by weight

Base:

1) distilled or deionized water—67.65%,

2) octoxynol-9-Triton® X-100 from Dow Chemical USA, 2.00%,

3) lauryl alcohol—CO-1214 from Procter & Gamble, 0.75%,

4) propylene glycol—propylene glycol USP from Llyondell Chemical Co., 5.00%,

5) dimethicone copolyol—Dow Corning® 190 SURFACTANT from Dow Corning Corp., 1.00%,

6) ethanol denat. (190 proof)—ethanol SD 40 (190), 2.00%,

7) dihydroxyacetone (75% active)—from EMD Chemicals Inc., 16.00%,

8) citric acid—citric acid USP from Dow Chemical USA, amount varies and adjusted to obtain pH of 2.4 to 2.8,

9) methylparaben—CoSept™ M from RTD-HallStar Inc., 0.20%,

10) propylparaben—CoSept™ P from RTD-HallStar Inc., 0.10%,

11) caramel—0.20%,

12) fragrance—SM 79207M from Shaw Mudge Co., 0.10%, and

13) distilled or deionized water—amount needed to obtain 100% weight.

The Base is mixed and prepared in a similar manner to Example 1.

Accelerator:

1) distilled or deionized water—82.40%,

2) acrylate/aminoacrylates/C10-30 alkyl PEG-20 (polyethylene glycol-20) itaconate copolymer—Structure Plus® 2001 (25-3802) from National Starch & Chemical Co., 10.00%,

3) glycine—from Aninimoto USA, Inc., 1.00%,

4) nonoxynol-9—Igepal CO-630™ from Rhodia Inc., 0.50%,

5) methylparaben—CoSept™ M from RTD-HallStar Inc., 0.20%,

6) propylparaben—CoSept™ P from RTD-HallStar Inc., 0.10%,

7) cetyl alcohol—LANETTE 16™ from Cognis Corporation, 0.30%,

8) decyl glucoside—PLANTAREN PS-100™ from Cognis Corporation, 0.50%, and

9) citric acid and/or triethanolamine—amount used to adjust the pH to 7.3, +/−0.3.

The Accelerator is prepared in a similar manner as that described in Example 1. The Base and the Accelerator are stored and dispensed in a similar manner to that in Example 1.

### EXAMPLE 4

An exothermic skin conditioning gel is prepared from the following Base and Developer. The listed concentration is percent by weight

Base:

1) distilled or deionized water-54.13%,

2) ceteareth-25—CREMOPHOR A 25™ from BASF Corporation, 12.00%,

3) lauryl alcohol—CO-1214 from Procter & Gamble, 0.75%,

4) PEG-25 (polyethylene glycol-25) hydrogenated castor oil—ARLATONE G™ from Uniqema Co., 1.50%,

5) propylene glycol—propylene glycol, USP from Lyondell Chemical Co., 5.00%,

6) monoethanolamine sulfite—THIOSET M™ from Dow Chemical, 14.26%,

7) cocamidopropyl betaine—MONATERIC CAB™ from Uniqema Americas, 4.00%,

8) triethanolamine—from Dow Chemical, amount necessary to obtain pH of 8.5+/−0.2,

9) DMDM hydantoin—GLYDANT™ from Lonza, Inc., 0.30%,

10) fragrance—0.06%, and

11) distilled or deionized water—amount necessary to achieve 100%.

The Base is mixed and prepared in a similar manner to Example 1.

Developer:

1) distilled or deionized water—50.00%,

2) 6% hydrogen peroxide (v/v)—hydrogen peroxide and water from FMC Corporation Pharmaceutical Div., 40.00%, and

3) acrylate/steareth-20 itaconate copolymer—STRUCTURE® 2001 from National Starch & Chemical Co., 10.00%.

The Developer is combined in a similar manner to that described in Example 1. The Base and the Developer are stored and dispensed in a similar manner to that in Example 1.

### EXAMPLE 5

An exothermic post-foaming hair conditioning and rinsing gel is prepared from the following Base and Developer. The listed concentration is percent by weight

Base:

1) distilled or deionized water—64.55%,

2) centrimonium chloride—VARISOFT 300™ from Degussa Co., 2.00%,

3) polyquaternium-10—UCARE POLYMER JR-400™ from Dow Chemical USA, 0.500%,

4) PEG-7 glyceryl cocoate—Cetiol HE™ from Cognis Corporation, 1.50%

5) quaternized wheat protein—MACKPRO NLW™ from McIntyre Group Ltd., 0.1%,

6) lauryl alcohol—CO-1214 from Procter & Gamble, 0.75%,

7) propylene glycol—propylene glycol USP from Lyondell Chemical Co., 5.00%,

8) monoethanolamine sulfite—THIOSET™ from Dow Chemical USA, 14.26%,

9) triethanolamine—from Dow Chemical, amount necessary to obtain pH of 9.0+/−0.2,

10) DMDM hydantoin—GLYDANT™ from Lonza, Inc., 0.30%,

11) fragrance—0.4%, and

12) distilled or deionized water—4.34%.

The Base is mixed and produced in a similar manner to Example 1.

Developer:

1) distilled or deionized water—50.00%,

2) 6% hydrogen peroxide (v/v)—hydrogen peroxide and water from FMC Corporation Pharmaceutical Div., 40.00%, and

3) acrylate/steareth-20 itaconate copolymer—Structure® 2001 from National Starch & Chemical Co., 10.00%.

The Developer is prepared in a similar manner to that described in Example 1. The Base and the Developer are stored and dispensed in a similar manner to that in Example 1.

### EXAMPLE 6

Representative post foaming shaving/utility gels that do not have hydrogen peroxide are set forth in the following tables.

TABLE 1
SPD EXOTHERMIC POST FOAMING SHAVING/UTILITY GEL E:

| | | | |
|---|---|---|---|
| INCI DESIGNATION | TRADE NAME | SUPPLIER | % by Wt |

PART A: UTILITY CREAM/GEL BASE

| | | | |
|---|---|---|---|
| 1,2 Propanediol | Propylene Glycol | Lyondell | (2) 46.64 |
| Cetyl Alcohol | Lanette 16 | Cognis | 0.30 |
| PPG-12-PEG-50-Lanolin | Lanoil AWS | Lanaetex | 2.00 |
| PPG-28-Buteth-35 | UCON 50-HB-3520 | Dow | 40.00 |
| Polyethylene Glycol | Carbowax 400 | Dow | 3.5 |
| 400NF | | | |
| Simmondsia Chinensis | Lipovol J | Lipo | 1.5 |
| (Jojoba) Seed Oil | | | |
| Triethanolamine | Tealan 99% | Rita | (1) 6.00 |
| Fragrance | Bell 6106533 | Bell | 0.06 |
| Total | | | 100.00 |

(1) q.s. to pH 9.0 +/− 0.2. Typical value 6.30
(2) q.s. to 100.0%

PART B: DEVELOPER:

| | | | |
|---|---|---|---|
| Water | Dist Water/ | | 83.10 |
| | DI Water | | |
| Phosphoric Acid | USP | General | (1) 0.50 |
| Nonoxynol-4 | Igepal CO 430 | Rhodia | 2.00 |
| Nonoxynol-9 | Igepal CO 630 | Rhodia | 2.00 |
| Decyl Glucoside | Plantaren 200N | Cognis | 2.00 |
| DMDM Hydantoin | Mackstat DM | McIntyre | 0.40 |
| Acrylates/Steareth-20 | Structure 2001 | National | 10.00 |
| Itaconate Copolymer | | Starch | |
| Total | | | 100.00 |

(1) q.s. to pH 3.0-4.0

TABLE 2
SPD EXOTHERMIC POST FOAMING SHAVING/UTILITY GEL A:

| | | | |
|---|---|---|---|
| INCI DESIGNATION | TRADE NAME | SUPPLIER | % by Wt |

PART A: UTILITY CREAM/GEL BASE

| | | | |
|---|---|---|---|
| 1,2 Propanediol | Propylene Glycol | Lyondell | 2) 43.64 |
| Cetyl Alcohol | Lanette 16 | Cognis | 0.30 |
| PEG/PPG-17/ | UCON 75-H-450 | Dow | 25.00 |
| 6 Coploymer | | | |
| Polyalkylene glycol | UCON Lubricant | Dow | 25.00 |
| momobutyl ether | 50-HB-3520 | | |
| Tromethamine | Tris Amino Ultra PC | Angus/Dow | (1) 6.00 |
| Fragrance | Bell 6106533 | Bell | 0.06 |
| Total | | | 100.0 |

PART B: DEVELOPER:

| | | | |
|---|---|---|---|
| Water | Dist Water/DI Water | | 83.10 |
| Phosphoric Acid | USP | General | (1) 0.50 |
| Nonoxynol-4 | Igepal CO 430 | Rhodia | 2.00 |
| Nonoxynol-9 | Igepal CO 630 | Rhodia | 2.00 |
| Decyl Glucoside | Plantaren 200N | Cognis | 2.00 |
| DMDM Hydantoin | Mackstat DM | McIntyre | 0.40 |
| Acrylates/Steareth-20 | Structure 2001 | National | 10.00 |
| Itaconate Copolymer | | Starch | |
| Total | | | 100.00 |

(1) q.s. to pH 3.0-4.0
(3) q.s. to pH 9.0 +/− 0.2. Typical value 6.30
(4) q.s. to 100.0%

TABLE 3
SPD EXOTHERMIC POST FOAMING SHAVING/UTILITY GEL B:

| | | | |
|---|---|---|---|
| INCI DESIGNATION | TRADE NAME | SUPPLIER | % by Wt |

PART A: UTILITY CREAM/GEL BASE

| | | | |
|---|---|---|---|
| 1,2 Propanediol | Propylene Glycol | Lyondell | 2) 43.64 |
| Cetyl Alcohol | Lanette 16 | Cognis | 0.30 |
| PEG/PPG-17/ | UCON 75-H-450 | Dow | 25.00 |
| 6 Coploymer | | | |
| Polyalkylene glycol | UCON Lubricant | Dow | 25.00 |
| momobutyl ether | 50-HB-3520 | | |
| Tromethamine | Tris Amino Ultra PC | Angus/Dow | (1) 6.00 |
| Fragrance | Bell 6106533 | Bell | 0.06 |
| Total | | | 100.00 |

PART B: DEVELOPER:

| | | | |
|---|---|---|---|
| Water | Dist Water/DI Water | | 83.10 |
| Phosphoric Acid | USP | General | (1) 0.50 |
| Nonoxynol-4 | Igepal CO 430 | Rhodia | 2.00 |
| Nonoxynol-9 | Igepal CO 630 | Rhodia | 2.00 |
| Decyl Glucoside | Plantaren 200N | Cognis | 2.00 |
| DMDM Hydantoin | Mackstat DM | McIntyre | 0.40 |
| Acrylates/Steareth-20 | Structure 2001 | National | 10.00 |
| Itaconate Copolymer | | Starch | |
| Total | | | 100.00 |

(1) q.s. to pH 3.0-4.0
(5) q.s. to pH 9.0 +/− 0.2. Typical value 6.30
(6) q.s. to 100.0%

TABLE 4
SPD EXOTHERMIC POST FOAMING SHAVING/UTILITY GEL D:

| | | | |
|---|---|---|---|
| INCI DESIGNATION | TRADE NAME | SUPPLIER | % by Wt |

PART A: UTILITY CREAM/GEL BASE

| | | | |
|---|---|---|---|
| 1,2 Propanediol | Propylene Glycol | Lyondell | 2) 33.94 |
| Cetyl Alcohol | Lanette 16 | Cognis | 0.30 |
| PEG/PPG-17/ | UCON 75-H-450 | Dow | 20.00 |
| 6 Coploymer | | | |
| Polyalkylene glycol | UCON Lubricant | Dow | 20.00 |
| momobutyl ether | 50-HB-3520 | | |
| Carbowax 400 | Polyethylene Glycol | Dow | 10.00 |
| | 400NF | | |
| Aluminum Silicate | Sylobloc K300 | Grace | 10.00 |
| | | Davison | |
| Tromethamine | Tris Amino Ultra PC | Angus/Dow | (1) 6.00 |
| Fragrance | Bell 6106533 | Bell | 0.06 |
| Total | | | 100.00 |

PART B: DEVELOPER:

| | | | |
|---|---|---|---|
| Water | Dist Water/DI Water | | 83.10 |
| Phosphoric Acid | USP | General | (1) 0.50 |
| Nonoxynol-4 | Igepal CO 430 | Rhodia | 2.00 |
| Nonoxynol-9 | Igepal CO 630 | Rhodia | 2.00 |
| Decyl Glucoside | Plantaren 200N | Cognis | 2.00 |
| DMDM Hydantoin | Mackstat DM | McIntyre | 0.40 |
| Acrylates/Steareth-20 | Structure 2001 | National | 10.00 |
| Itaconate Copolymer | | Starch | |
| Total | | | 100.00 |

(1) q.s. to pH 3.0-4.0
(7) q.s. to pH 9.0 +/− 0.2. Typical value 6.30
(8) q.s. to 100.0%

TABLE 5
SPD EXOTHERMIC POST FOAMING SHAVING/UTILITY GEL D1:

| | | | |
|---|---|---|---|
| INCI DESIGNATION | TRADE NAME | SUPPLIER | % by Wt |

PHASE A: UTILITY CREAM/GEL BASE

| | | | |
|---|---|---|---|
| 1,2 Propanediol | Propylene Glycol | Lyondell | 2) 50.14 |
| Citric Acid | Citric Acid, USP | General | (1) 2.5 |
| Cetyl Alcohol | Lanette 16 | Cognis | 0.30 |
| PPG-12-PEG-50-Lanolin | Lanoil AWS | Lanaetex | 2.00 |
| PPG-28-Buteth-35 | UCON 50-HB-3520 | Dow | 40.00 |
| Polyethylene Glycol | Carbowax 400 | Dow | 3.5 |
| 400NF | | | |
| Simmondsia Chinensis | Lipovol J | Lipo | 1.5 |
| (Jojoba) Seed Oil | | | |
| Fragrance | Bell 6106533 | Bell | 0.06 |
| Total | | | 100.00 |

PART B: DEVELOPER:

| | | | |
|---|---|---|---|
| Water | Dist Water/DI | | 72.10 |
| | Water | | |
| Nonoxynol-4 | Igepal CO 430 | Rhodia | 2.00 |
| Nonoxynol-9 | Igepal CO 630 | Rhodia | 2.00 |
| Decyl Glucoside | Plantaren 200N | Cognis | 2.00 |
| DMDM Hydantoin | Mackstat DM | McIntyre | 0.40 |
| Acrylates/ | Structure Plus | National | 20.00 |
| Aminoacrylates/C10-30 | | | |
| Alkyl PEG-20 | | Starch | |
| Itaconate Copolymer | | | |
| 2-Amino-2-methyl-1- | AMP-95 | Angus/Dow | (1) 1.5 |
| propanol | | | |
| Total | | | 100.00 |

(1) q.s. to pH 7.5-8.5
(9) q.s. to pH 4.0 +/− 0.5.
(10) q.s. to 100.0%

What is claimed is:

1. A viscous product for use on the body comprising a first low viscosity component formulation and a second low viscosity component formulation.

2. The viscous product of claim 1 wherein said viscous product is heated upon reacting said first low viscosity component formulation with said second low viscosity component formulation.

3. The viscous of claim 1 wherein said viscous product is post-foaming.

4. The viscous product of claim 1 wherein said viscous product is formulated for shaving, body washing, tanning, skin or hair conditioning.

5. The viscous product of claim 4 wherein said viscous product is formulated for shaving, said first low viscosity component formulation comprises a solvent, a thickener, a substance that generates heat upon dissolution in water and a substance that increases and buffers pH, and said second low viscosity component formulation contains water, an acidifier, a solubilizer or surfactant and a thickener.

6. The viscous product of claim 4 wherein said viscous product is formulated for shaving, said first low viscosity component formulation comprises water, sodium lauryl sulfate, lauryl alcohol, propylene glycol, monoethanolamine sulfite, oleamine oxide, triethanolamine, fragrance, and a preservative, and said second low viscosity component formulation comprises water, hydrogen peroxide and an acrylate/steareth-20 itaconate copolymer.

7. The viscous product of claim 6 wherein the concentration in weight percent of the sodium lauryl sulfate is from 0.5% to 90.0%, the lauryl alcohol is from 0.1% to 7.0%, the propylene glycol is from 0.1% to 15.0%, the monoethanolamine sulfite is from 0.5% to 30.0%, the oleamine oxide is from 0.1% to 15.0%, the triethanolamine is from 1.0% to 15.0%, the fragrance is from 0.005% to 3.0%, the preservative is from 0.2% to 1.2%, the hydrogen peroxide is from 10.0% to 80.0% and the acrylate/steareth-20 itaconate copolymer is from 2.0% to 30.0%.

8. The viscous product of claim 6 wherein the concentration in weight percent of the sodium lauryl sulfate is from 9.0% to 20.0%, the lauryl alcohol is from 0.2% to 2.0%, the propylene glycol is from 3.0% to 7.0%, the monoethanolamine sulfite is from 10.0% to 20.0%, the oleamine oxide is from 1.0% to 8.0%, the triethanolamine is from 3.0% to 8.0%, the fragrance is from 0.01% to 1.5%, the hydrogen peroxide is from 25.0% to 55.0% and the acrylate/steareth-20 itaconate copolymer is from 5.0% to 25.0%.

9. The viscous product of claim 4 wherein said viscous product is formulated for body washing, said first low viscosity component formulation comprises water, sodium lauryl sulfate, sodium laureth sulfate, lauryl alcohol, glycerin, monoethanolamine sulfite, cocamidopropyl betaine, ethanol, polyquaternium-10, triethanolamine, DMDM hydantoin and a fragrance, and said second low viscosity component formulation comprises water, hydrogen peroxide, an acrylate/steareth-20 iconate copolymer, nonoxynol-4, nonoxynol-5, cetyl alcohol and decyl glucoside.

10. The viscous product of claim 9 wherein the weight concentration of the sodium lauryl sulfate is from 0.5% to 90.0%, the sodium laureth sulfate is from 0.5% to 90.0%, the lauryl alcohol is from 0.1% to 7.0%, the glycerin is from 0.1% to 15.0%, the monoethanolamine sulfite is from 0.5% to 30.0%, the cocamidopropyl betaine is from 0.1% to 15.0%, the ethanol is from 0.1% to 7.5%, of the polyquatemium-10 is from 0.05% to 2.5%, the triethanolamine is from 1.0% to 15.0%, the DMDM hydantoin is from 0.05% to 2.0%, the fragrance is from 0.005% to 3.0%, the hydrogen peroxide is from 10.0% to 80.0%, the acrylates/steareth-20 iconate copolymer is from 2.0% to 30.0%, the nonoxynol-4 is from 0.05% to 5.0%, the nonoxynol-5 is from 0.05% to 5.0%, the cetyl alcohol is from 0.05% to 5.0% and the decyl glucoside is from 0.05% to 5.0%.

11. The viscous product of claim 10 wherein the weight concentration of the sodium lauryl sulfate is from 9.0% to 20.0%, the sodium laureth sulfate is from 9.0% to 20.0%, the lauryl alcohol is from 0.2% to 2.0%, the glycerin is from 3.0% to 7.0%, the monoethanolamine sulfite is from 10.0% to 20.0%, the cocamidopropyl betaine is from 1.0% to 8.0%, the ethanol is from 0.5% to 5.0%, the polyquaternium-10 is from 0.05% to 1.0%, the triethanolamine is from 3.0% to 8.0%, the DMDM hydantoin is from 0.1% to 1.0%, the fragrance is from 0.01% to 1.5%, the hydrogen peroxide is from 25.0% to 55.0%, the acrylates/steareth-20 iconate copolymer is from 5.0% to 25.0%, the nonoxynol-4 is from 0.10% to 3.0%, the nonoxynol-5 is from 0.10% to 3.0%, the cetyl alcohol is from 0.10% to 3.0% and the decyl glucoside is from 0.10% to 3.0%.

12. The viscous product of claim 4 wherein said viscous product is formulated for tanning, said first low viscosity component formulation comprises water, octoxynol-9, lauryl alcohol, propylene glycol, dimethicone copolyol, ethanol, dihydroxyacetone, citric acid, methylparaben, propylparaben, caramel and a fragrance, and said second low viscosity component formulation comprises water, acrylates/aminoacrylates/C10-30 alkyl polyethylene glycol-20 itaconate copolymer, glycine, nonoxynol-9, methylparaben, propylparaben, cetyl alcohol, decyl glucoside and citric acid/triethanolamine.

13. The viscous product of claim 12 wherein for said first low viscosity component formulation, the weight percent of the octoxynol-9 is 2.00%, the lauryl alcohol is from 0.1% to 7.0%, the propylene glycol is from 0.1% to 15.0%, the dimethicone copolyol is 1.00%, the ethanol is from 0.1% to 7.5%, the dihydroxyacetone is from 2.0% to 22.0%, the citric acid is from 0.01% to 5.0%, the methylparaben is from 0.02% to 1.0%, the propylparaben is from 0.02% to 1.0%, the caramel is from 0.01% to 4.0% and the fragrance is from 0.005% to 4.0%, and for said second low viscosity component formulation, the weight percent of the acrylate/aminoacrylate/C10-30 alkyl polyethylene glycol-20 itaconate copolymer is from 2.0% to 30.0%, the glycine is from 0.1% to 15.0%, nonoxynol-9 is from 0.05% to 5.0%, the methylparaben is from 0.02% to 1.0%, the propylparaben is from 0.02% to 1.0%, the cetyl alcohol is from 0.05% to 5.0% and the decyl glucoside is from 0.05% to 5.0%.

14. The viscous product of claim 13 wherein for said first low viscosity component formulation, the weight percent of the lauryl alcohol is from 0.2% to 2.0%, the propylene glycol is from 3.0% to 7.0%, the ethanol is from 0.5% to 5.0%, of the dihydroxyacetone is from 6.0% to 18.0%, the citric acid is from 0.05% to 1.5%, of the methylparaben is from 0.05% to 1.0%, the propylparaben is from 0.05% to 1.0%, the caramel is from 0.10% to 2.0% and the fragrance is from 0.05% to 1.5%, and for said second low viscosity component formulation, the weight percent of the acrylate/aminoacrylate/C10-30 alkyl polyethylene glycol-20 itaconate copolymer is from 5.0% to 25.0%, the glycine is from 1.0% to 5.0%, the nonoxynol-9 is from 0.10% to 3.0%, the methylparaben is from 0.05% to 1.0%, the propylparaben is from 0.05% to 1.0%, the cetyl alcohol is from 0.10% to 3.0% and the decyl glucoside is from 0.10% to 3.0%.

15. The viscous product of claim 4 wherein said viscous product is formulated for skin conditioning, said first low viscosity component formulation comprises water, ceteareth-25, lauryl alcohol, polyethylene glycol-25 hydrogenated castor oil, propylene glycol, monoethanolamine sulfite, cocamidopropyl betaine, triethanolamine, DMDM hydantoin and a fragrance, and said second low viscosity component formulation comprises water, hydrogen peroxide and acrylates/steareth-20 itaconate copolymer.

16. The viscous product of claim 15 wherein the weight percent of the ceteareth-25 is from 5.0% to 25.0%, the lauryl alcohol is from 0.1% to 7.0%, the PET-25 hydrogenated castor oil is from 0.2% to 10.0%, the propylene glycol is from 0.1% to 15.0%, the monoethanolamine sulfite is from 0.5% to 30.0%, the cocamidopropyl betaine is from 0.1% to 15.0%, the triethanolamine is from 1.0% to 15.0%, the DMDM hydantoin is from 0.05% to 2.0%, the fragrance is from 0.005% to 3.0%, the hydrogen peroxide is from 10.0% to 80.0% and the acrylate/steareth-20 itaconate copolymer is from 2.0% to 30.0%.

17. The viscous product of claim 15 wherein the weight percent of the ceteareth-25 is from 9.0% to 18.0%, the lauryl alcohol is from 0.2% to 2.0%, the polyethylene glycol-25 hydrogenated castor oil is from 0.5% to 5.0%, the propylene glycol is from 3.0% to 7.0%, the monoethanolamine sulfite is from 10.0% to 20.0%, the cocamidopropyl betaine is from 1.0% to 8.0%, the triethanolamine is from 3.0% to 8.0%, the DMDM hydantoin is from 0.10% to 1.0%, the fragrance is from 0.01% to 1.5%, the hydrogen peroxide is from 25.0% to 55.0% and the acrylate/steareth-20 itaconate copolymer is from 5.0% to 25.0%.

18. The viscous product of claim 4 wherein said viscous product is formulated for hair conditioning and rinsing, said first low viscosity component formulation comprises water, cetrimonium chloride, polyquaternium-10, polyethylene glycol-7 glyceryl cocoate, quaternized wheat protein, lauryl alcohol, propylene glycol, monoethanolamine sulfite, triethanolamine, DMDM hydantoin, and a fragrance, and said second low viscosity component formulation comprises water, hydrogen peroxide and an acrylate/steareth-20 itaconate copolymer.

19. The viscous product of claim 18 wherein the weight percent of the cetrimonium chloride is from 0.1% to 8.0%, the polyquaternium-10 is from 0.05% to 2.5%, the polyethylene glycol-7 glyceryl cocoate is from 0.10% to 7.5%, the quaternized wheat protein is from 0.05% to 10.0%, the lauryl alcohol is from 0.1% to 7.0%, the propylene glycol is from 0.1% to 15.0%, the monoethanolamine sulfite is from 0.5% to 30.0%, the triethanolamine is from 1.0% to 15.0%, the DMDM hydantoin is from 0.05% to 2.0%, the fragrance is from 0.05% to 2.0%, the hydrogen peroxide is from 10.0% to 80.0% and the acrylate/steareth-20 itaconate copolymer is from 2.0% to 30.0%.

20. The viscous product of claim 19 wherein the weight percent of the cetrimonium chloride is from 0.5% to 5.0%, of the polyquaternium-10 is from 0.05% to 1.0%, the polyethylene glycol-7 glyceryl cocoate is from 0.2% to 4.0%, the quaternized wheat protein is from 0.1% to 5.0%, the lauryl alcohol is from 0.2% to 2.0%, the propylene glycol is from 3.0% to 7.0%, the monoethanolamine sulfite is from 10.0% to 20.0%, the triethanolamine is from 3.0% to 8.0%, the DMDM hydantoin is from 0.1% to 1.0%, the fragrance is from 0.1% to 1.0%, of the hydrogen peroxide is from 25.0% to 55.0% and the acrylate/steareth-20 itaconate copolymer is from 5.0% to 25.0%.

21. A dispenser containing the viscous product of claim 4.
